(19) United States
(12) Patent Application Publication
(10) Pub. No.: US 2005/0031638 A1
(43) Pub. Date: Feb. 10, 2005

(54) Vaccine

(75) Inventors: Wilfried L J. Dalemans, Hoegaarden (BE); Catherine Marie Ghislaine Gerard, Rhode Saint Genese (BE)

Correspondence Address: GLAXOSMITHKLINE;Corporate Intellectual Property - UW2220, P.O. Box 1539, King of Prussia, PA, 19406-0939, US

(73) Assignee: SmithKline Beecham Biologicals s.a.

(21) Appl. No.: 10/899,771

(22) Filed: Jul. 27, 2004

Related U.S. Application Data
(63) Continuation of application No. 09/581,976, filed on Jun. 20, 2000, now abandoned.

(30) Foreign Application Priority Data
Dec. 24, 1997 (GB) 9727262.9

(51) Int. Cl. A61K 48/00; A61K 39/12

(52) U.S. Cl. 424/186.1; 514/44

(57) ABSTRACT

The present invention provides Human Papilloma Virus (HPV) fusion proteins, linked to an immunological fusion partner that provides T helper epiptopes to the HPV antigen. Vaccine formulations are provided that are useful in the treatment or Prophylaxis of HPV induced tumours.

The present invention relates to vaccine compositions, comprising an E6 or/and E7 or E6, E7 fusion protein from an HPV strain optionally linked with an immunological fusion partner and formulated with a CpG containing oligonucleotide into vaccines that find utility in the treatment or prophylaxis of human papilloma virus induced tumours or lesions. In particular, the present invention relates to vaccines comprising fusions proteins, comprising a protein or part of a protein that provides T helper epitopes (such as protein D from Haemophilus influenzae B) and an antigen from a human-papilloma virus (eg comprising an E6 or E7 protein from HPV 16 or 18 strain associated with cancer) that find utility in the treatment or prophylaxis of human papilloma induced tumours, wherein the vaccine is formulated with a CpG containing oligonucleotide as an adjuvant.

Papillomaviruses are small naked DNA tumour viruses (7.9 kilobases, double strand), which are highly species-specific. Over 70 individual human papillomavirus (HPV) genotypes have been described. Papillomaviruses are classified on the basis of species of origin (human, bovine etc.) and of the degree of genetic relatedness with other papillomaviruses from the same species. HPVs are generally specific for the skin or mucosal surfaces and have been broadly classified into “low” and “high” risk viruses.

Low risk HPVs usually cause benign lesions (warts or papillomas) that persist for several months or years. High risk HPVs are associated with pre-neoplastic lesions and cancer. The strongest positive association between an HPV virus and human cancer is that which exist between HPV 16 and 18 and cervical carcinoma. More than ten other HPV types have also been found in cervical carcinomas including HPV 31 and HPV 33 although at less frequency.

Genital HPV infection in young sexually active women is common and most individuals either clear the infection, or if lesions develop, these regress. Only a subset of infected individuals has lesions which progress to high grade intraephithelial neoplasia and only a fraction of these progress further to invasive carcinoma.

The molecular events leading to HPV infection have not been clearly established. The lack of an adequate in vitro system to propagate human papillomaviruses has hampered the progress to a best information about the viral cycle.

Today, the different types of HPVs have been isolated and characterised with the help of cloning systems in bacteria and more recently by PCR amplification. The molecular organisation of the HPV genomes has been defined on a comparative basis with that of the well characterised bovine papillomavirus type 1 (BPV1).

Although minor variations do occur, all HPVs genomes described have at least seven early genes, E1 to E7 and two late genes L1 and L2. In addition, an upstream regulatory region harbors the regulatory sequences which appears to control most transcriptional events of the HPV genome.

E1 and E2 genes are involved in viral replication and transcriptional control, respectively and tend to be disrupted by viral integration. E6 and E7 are involved in viral transformation. E5 has also been implicated in this process.

In the HPVs involved in cervical carcinoma such as HPV 16 and 18, the oncogenic process starts after integration of viral DNA. The integration results in the inactivation of genes coding for the capsid proteins L1 and L2 and loss of E2 repressor function leads to deregulation of the E6/E7 open reading frame installing continuously overexpression of the two early proteins E6 and E7 that will lead to gradually loss of the normal cellular differentiation and the development of the carcinoma. E6 and E7 overcome normal cell cycle by inactivating major tumor suppressor proteins, p53 and pRB, the retinoblastoma gene product, respectively.

Carcinoma of the cervix is common in women and develops through a pre-cancerous intermediate stage to the invasive carcinoma which frequently leads to death. The intermediate stages of the disease is known as cervical intraepithelial neoplasia and is graded I to III in terms of increasing severity (CIN I-III).

Clinically, HPV infection of the female anogenital tract manifests as cervical flat condylomas, the hallmark of which is the koilocytosis affecting predominantly the superficial and intermediate cells of the cervical squamous epithelium.

Koilocytes which are the consequence of a cytopathic effect of the virus, appear as multinucleated cells with a perinuclear clear haloe. The epithelium is thickened with abnormal keratinisation responsible for the warty appearance of the lesion.

Such flat condylomas when positive for the HPV 16 or 18 serotypes, are high-risk factors for the evolution toward cervical intraepithelial neoplasia (CIN) and carcinoma in situ (CIS) which are themselves regarded as precursor lesions of invasive cervix carcinoma.

The natural history of oncogenic HPV infection presents three consecutive phases, namely:
(1) a latent infection phase,
(2) a phase of intranuclear viral replication with product of complete virions, which corresponds to the occurrence of koilocytes. At this stage, the HPV is producing its full range of proteins including E2, E5, E6, E7, L1 and L2.
(3) a phase of viral integration into the cellular genome, which triggers the onset of malignant transformation, and corresponds to CIN II and CIN III/CIS with progressive disappearance of koilocytes. At this stage, the expression of E2 is down-regulated, the expression of E6 and E7 is enhanced. Between CIN II/III and CIN III/Cervix carcinoma the viral DNA changes from being episomal in the basal cells to integration of E6 and E7 genes only (tumoral cells). 85% of all cervix carcinomas are squamous cell carcinomas most predominantly related to the HPV 16 serotype. 10% and 5% are adenocarcinomas and adenosquamous cell carcinomas respectively, and both types are predominantly related to HPV 18 serotype. Nevertheless other oncogenic HPV's exist.

International Patent Application No. WO 96/19496 discloses variants of human papilloma virus E6 and E7 proteins, particularly fusion proteins of E6/E7 with a deletion in both the E6 and E7 proteins. These deletion fusion proteins are said to be immunogenic.

Immunomodulatory oligonucleotides contain unmethylated CpG dinucleotides (“CpG”) and are known (WO 96/02555. EP 468520). CpG is an abbreviation for cytosine-guanosine dinucleotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, Nature 374, p546 1995. Detailed analysis has shown that the CG motif has to be in a certain sequence context, and that such sequences are common in bacterial DNA but are rare in vertebrate DNA.

It is currently believed that this evolutionary difference allows the vertebrate immune system to detect the presence of bacterial DNA (as occurring during an infection) leading consequently to the stimulation of the immune system. The immunostimulatory sequence as defined by Krieg is:

Purine Purine CG pyrimidine pyrimidine and where the CG motif is not methylated. In certain combinations of the six nucleotides a palindromic sequence is present. Several of these motifs, either as repeats of one motif or a combination of different motifs, can be present in the same oligonucleotide. The presence of one or more of these immunostimulatory sequence containing oligonucleotides can activate various immune subsets, including natural killer cells (which produce interferon γ and have cytolytic activity) and macrophages (Wooldrige et al Vol 89 (no. 8), 1977). Although other unmethylated CpG containing sequences not having this consensus sequence have now been shown to be immunomodulatory.

The present invention provides compositions comprising either an E6 or/and E7 or an E6/E7 fusion protein optionally linked to an immunological fusion partner having T cell epitopes, and adjuvanted with an immunomodulatory CpG containing oligonucleotide.

In a preferred form of the invention, the immunological fusion partner is derived from protein D of Heamophilus influenza B. Preferably the protein D derivative comprises approximately the first ⅓ of the protein, in particular approximately the first N-terminal 100-110 amino acids. The protein D may be lipidated (Lipo Protein D). Other immunological fusion partners include the non-structural protein from influenzae virus, NS1 (hemagglutinin). Typically the N terminal 81 amino acids are utilised, although different fragments may be used provided they include T-helper epitopes.

In another embodiment the immunological fusion partner is the protein known as LYTA. Preferably the C terminal portion of the molecule is used. Lyta is derived from Streptococcus pneumoniae which synthesize an N-acetyl-L-alanine amidase, amidase LYTA, (coded by the lytA gen {Gene, 43 (1986) page 265-272} an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at its amino terminus has been described {Biotechnology: 10. (1992) page 795-798}. As used herein a preferred embodiment utilises the repeat portion of the Lyta molecule found in the C terminal end starting at residue 178. A particularly preferred form incorporates residues 188-305.

Accordingly, the present invention in preferred embodiment provides compositions comprising an immunomodulatory CpG oligonucleotide and a fusion proteins comprising Protein D-E6 from HPV 16, Protein D-E7 from HPV 16 Protein D-E7 from HPV 18, Protein D-E6 from HPV 18, and Protein D E6 E7 from both HPV 16 and 18. The protein D part preferably comprises the first ⅓ of protein D. It will be appreciated that other E6 and E7 proteins may be utilised from other HPV subtypes.

The proteins utilised in the present invention preferably are expressed in E. coli. In a preferred embodiment the proteins are expressed with a Histidine tail comprising between 5 to 9 and preferably six Histidine residues. These are advantageous in aiding purification.

The protein E7 may in a preferred embodiment carry one or several mutations in the binding site for the rb (retinoblastoma gene product) and hence eliminate any potential transforming capacity. Preferred mutations for HPV 16 E7 involve replacing Cys₂₄ with Glycine, or Glutamic acid₂₆ with Glutamine. In a preferred embodiment the E7 protein contains both these mutations.

Preferred mutations for the HPV 18 E₇ involve replacing Cys₂₇ with Glycine and/or Glutamic acid₂₉ with Glutamine. Again preferably both mutations are present.

Single or double mutations may also be introduced p53 region of E₆ to eliminate any potential transforming ability.

In a further embodiment of the invention there is provided and E6 E7 fusion protein from HPV linked to an immunological fusion partner and a CpG immunomodulatory oligonucleotide.

The vaccine of the present invention preferentially induce a TH1 immune response.

Two main types of Helper T cells have been characterized TH1 and TH2, which differ in the type of cytokines they secrete. These cytokines can be considered as the driving force behind the development of 2 different types of immune response TH1-type of immune response is associated with cell mediated effector mechanisms such as production of the INF-γ and IL-2 cytokines by T-lymphocytes. INF-γ which in turn can activate other cells and induce them to secrete other important cytokines and mediators (INF-γ-activated NK cells produce IL12, IL2-activated NK cells are transformed into lymphokine activated killer cell (LAK), INF-γ-activated macrophages secrete inflamatory mediators like TNFa, IL1, IL6 and release nitric oxyde, IL2 can provide help for the differentiation of antigen specific, haplotype restricted cytotoxic T lymphocytes (CTL). At the antibody level, in mice, Th1-type of immune response is also associated with the generation of antibodies of the IgG2 isotype (IgG2a in Balb/c mice and IgG2b in C57BL/6 mice).

The Th2-type of immune response is associated with a humoral immune response to the antigen. with the production of cytokines like IL4, IL5, IL6, IL10 and by the generation of a broad range of immunoglobulin isotypes including in mice IgG1, IgA, and IgM.

In man the distinction of Th1 and Th2-type immune responses is not absolute.

An individual will support an immune response which is predominantly Th1 or predominantly Th2. However, it is often convenient to consider the families of cytokines in terms of that described in murine CD4+ve T cell clones by Mosmann and Coffman (Mosmann, T. R. and Coffman, R. L. (1989) TH1 and TH2 cells: different patterns of lymphokine secretion lead to different functional properties. Annual Review of Immunology, 7, p145-173).

In the human TH1 type of response is also associated with the presence of cytokine (IFNg and IL2) eventually with the presence of CT1 and IgG2 isotypes in mice correspond to IgG1 type antibodies

This type 1 phenotype is of particular importance in protecting against viral and intracellular bacterial infections as well as in the treatment of cancer.

To manufacture the proteins used in the invention by recombinant techniques, an expression strategy can be used which involves fusion of E7, E6 or E6/E7 fusion to the 1/3-N-terminal portion of protein D from Haemophilus influenzae B, an immunological fusion partner providing T cell helper epitopes. An affinity polyhistidine tail is engineered at the carboxy terminus of the fusion protein allowing for simplified purification. Such recombinant antigen is overexpressed in E. coli as insoluble protein.

The proteins of the invention my be coexpressed with thioredoxin in trans (TIT). Coexpression of thioredoxin in trans versus in cis is preferred to keep antigen free of thioredoxin without the need for protease. Thioredoxin coexpression eases the solubilisation of the proteins of the invention. Thioredoxin coexpression has also a significant impact on protein purification yield, on purified-protein solubility and quality.

The replicable expression vectors may be prepared in accordance with the invention, by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the DNA polymer may be preformed or formed during the construction of the vector, as desired.

The choice of vector will be determined in part by the host cell, which may be prokaryotic or eukaryotic but preferably is E. coli. Suitable vectors include plasmids, bacteriophages, cosmids and recombinant viruses.

The preparation of the replicable expression vector may be carried out conventionally with appropriate enzymes for restriction, polymerisation and ligation of the DNA, by procedures described in, for example. Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed. IRL Press Ltd, 1985.

The choice of transforming conditions is determined by the host cell. Thus, a bacterial host such as E. coli may be treated with a solution of CaCl₂ (Cohen et al., Proc. Nat. Acad. Sci., 1973, 69, 2110) or with a solution comprising a mixture of RbCl, MnCl₂, potassium acetate and glycerol, and then with 3-[N-morpholino]-propane-sulphonic acid, RbCl and glycerol. Mammalian cells in culture may be transformed by calcium co-precipitation of the vector DNA onto the cells. The invention also extends to a host cell transformed with a replicable expression vector of the invention.

Culturing the transformed host cell under conditions permitting expression of the DNA polymer is carried out conventionally, as described in, for example, Maniatis et al. and “DNA Cloning” cited above. Thus, preferably the cell is supplied with nutrient and cultured at a temperature below 50° C.

The product is recovered by conventional methods according to the host cell. Thus, where the host cell is bacterial, such as E. coli it may be lysed physically, chemically or enzymatically and the protein product isolated from the resulting lysate. Where the host cell is mammalian, the product may generally be isolated from the nutrient medium or from cell free extracts. Conventional protein isolation techniques include selective precipitation, adsorption chromatography, and affinity chromatography including a monoclonal antibody affinity column.

When the proteins of the present invention are expressed with a hisitidine tail (His tag). The proteins can easily be purified by affinity chromatography using an ion metal affinity chromatography column (IMAC) column.

A second chromatographic step, such as Q-sepharose may be utilised either before or after the IMAC column to yield highly purified protein. If the immunological fusion partner is C-LYTA, then it is possible to exploit the affinity of CLYTA for choline and/or DEAE to purify this product. Products containing both C-LYTA and his tags can be easily and efficiently purified in a two step process involving differential affinity chromatography. One step involves the affinity of the His tag to IMAC columns, the other involves the affinity of the C-terminal domain of LYTA for choline or DEAE.

A preferred vaccine composition comprises at least Protein D-E6 from HPV 16 or derivative thereof together with Protein D-E7 from HPV 16. Alternatively the E6 and E7 may be presented in a single molecule, preferably a Protein D E6/E7 fusion. Such vaccine may optionally contain either or both E6 and E7 proteins from HPV 18, preferably in the form of a Protein D-E6 or Protein D-E7 fusion protein or Protein D E6/E7 fusion protein. The vaccines of the present invention may contain other HPV antigens from HPV 16 or 18. In particular, the vaccine may contain L1 or L2 antigen monomers. Alternatively such L1 or L2 antigens may be presented together as a virus like particle or the L1 alone protein may be presented as virus like particle or caposmer structure. Such antigens, virus like particles and capsomer are per se known. See for example WO94/00152, WO94/20137, WO94/05792, and WO93/02184. Additional early proteins may be included such as E2 or preferably E5 for example The vaccine of the present invention may additionally comprise antigens from other HPV strains, preferably from strains HPV 6, 11, 31 or 33.

Vaccine preparation is generally described in Vaccine Design—The subunit and adjuvant approach (Ed. Powell and Newman) Pharmaceutical Biotechnology Vol. 6 Plenum Press 1995. Encapsulation within liposomes is described by Fullerton, U.S. Pat. No. 4,235,877.

The preferrred oligonucleotides preferably contain two or more CpG motifs separated by six or more nucleotides. The oligonucleotides of the present invention are typically deoxynucleotides. In a preferred embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages.

Preferred oligonucleotides have the following sequences: The sequences preferably contain all phosphorothioate modified internucleotide linkages.

| | |
|---|---|
| OLIGO 1: | TCC ATG ACG TTC CTG ACG TT |
| OLIGO 2: | TCT CCC AGC GTG CGC CAT |
| OLIGO 3: | ACC GAT GAC GTC GCC GGT GAC GGC ACC ACG |

The CpG oligonucleotides utilised in the present invention may be synthesized by any method known in the art (eg EP 468520). Conveniently, such oligonucleotides may be synthesized utilising an automated synthesizer. Methods for producing phosphorothioate oligonucleotides or phosphorodithioate are described in U.S. Pat. No. 5,666,153, U.S. Pat. No. 5,278,302 and WO95/26204.

The invention will be further described by reference to the following examples:

### EXAMPLE I

### Construction of an E. coli Strain Expressing Fusion Protein-D1/3-E7-His (HPV16)

1)—Construction of Expression Plasmid

a)—Plasmid pMG MCS prot D1/3 (=pRIT14589) is a derivative of pMG81 (described in UK patent application no 951 3261.9 published as WO97/01640) in which the codons 4-81 of NS1 coding region from Influenza were replaced by the codons corresponding to residues Ser 20→Thr127 of mature protein D of Haemophilus Influenzae strain 772, biotype 2 (H. Janson et al., 1991. Infection and Immunity, Jan. p.119-125). The sequence of Prot-D1/3 is followed by a multiple cloning site (11 residues) and a coding region for a C-terminal histidine tail (6 His). This plasmid is used to express the fusion protein D1/3-E7-His.
b)—HPV genomic E6 and E7 sequences type HPV 16 (See Dorf et al., Virology 1985, 145, p. 181-185) were amplified from HPV 16 full length genome cloned in pBR322 (obtained from Deutsches Krebsforschungszentrum (DKFZ), Referenzzentrum für human pathogen Papillomaviruses—D 69120—Heidelberg) and were subcloned into pUC19 to give TCA 301 (=pRIT14462).
   Construction of Plasmid TCA 308 (=pRIT14501): a Plasmid Expressing the Fusion Protein-D1/3-E7-His

The nucleotides sequences corresponding to amino acids 1→98 of E7 protein are amplified from pRIT14462. During the polymerase chain reaction, NcoI and SpeI restriction sites were generated at the 5′ and 3′ ends of the E7 sequences allowing insertion into the same sites of plasmid pMGMCS Prot D1/3 to give plasmid TCA308 (=pRIT14501). The insert was sequenced to verify that no modification had been generated during the polymerase chain reaction. The sequence for the fusion protein-D1/3-E7-His (HPV 16) is described in sequence ID No.1 and the coding sequence in ID No.2.

2)—Transformation of AR58 Strain

Plasmid pRIT14501 was introduced into E. coli AR58 (Mott et al., 1985, Proc. Natl. Acad. Sci., 82:88) a defective λ lysogen containing a thermosensitive repressor of the λ pL promoter.

3)—Growth and Induction of Bacterial Strain—Expression of Prot-D1/3-E7-His

Cells of AR58 transformed with plasmid pRIT14501 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein D1/3-E7-His. The incubation at 39° C. was continued for 4 hours. Bacteria were pelleted and stored at −20° C.

### EXAMPLE II

### Construction of an E. coli Strain Expressing Fusion Protein-D1/3-E6-his/HPV16

1. Construction of Expression Plasmid

a) Plasmid pMG MCS prot D1/3 (=pRIT14589) is a derivative of pMG81 (described in WO97/01640 in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p. 119-125). The sequence of Prot-D1/3 is followed by a multiple cloning site (11 residues) and a coding region for a C-terminal histidine tail (6 His). This plasmid is used to express the fusion protein D1/3-E6-his.
b) HPV genomic E6 and E7 sequences type HPV16 (Seedorf et al., Virology 1985, 145, p.181-185) were amplified from HPV16 full length genome cloned in pBR322 (obtained from Deutsches Krebsforschungszentrun (DKFZ), Referenzzentrum für human pathogen Papillomaviruses—
c) D 69120—Heidelberg) and were subcloned into pUC19 to give TCA 301 (=pRIT14462).
   Construction of Plasmid TCA 307 (=pRIT14497): a Plasmid Expressing the Fusion Protein-D1/3-E6-His/HPV16

The nucleotides sequences corresponding to amino acid.

1→151 of E6 protein were amplified from pRIT14462. During the polymerase chain reaction, NcoI and SpeI restriction sites were generated at the 5′ and 3′ ends of the E6 sequences allowing insertion into the same sites of plasmid pMGMCS Prot D1/3 to give plasmid TCA307 (=pRIT14497). The insert was sequenced to verify that no modification had been generated during the polymerase chain reaction. The protein and coding sequence for the fusion protein-D1/3-E6-His is described in sequence ID No.3 and 4.

2. Transformation of AR58 Strain

Plasmid pRIT14497 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of Prot-D113-E6-His

Cells of AR58 transformed with plasmid pRIT14497 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein D1/3-E6-his. The incubation at 39° C. was continued for 4 hours. Bacteria were pelleted and stored at −20C.

4. Characterization of Fusion Protein D1/3-E6-his (HPV 16)

Preparation of Extracts

Frozen cells are thawed and resuspended in 10 ml of PBS buffer. Cells are broken in a French pressure cell press SLM Aminco at 20,000 psi (three passages). The extract is centrifuged at 16,000 g for 30 minutes at 4° C.

Analysis on Coomassie-Stained SDS-Polyacrylamide Gels and Western Blots

After centrifugation of extracts described above, aliquots of supernatant and pellet were analysed by SDS-polyacrylamide gel electrophoresis and Western blotting.

A major band of about 32 kDa, localized in the pellet fraction, was visualised by Coomassie stained gels and identified in Western blots by rabbit polyclonal anti-protein-D and by Ni-NTA conjugate coupled to calf intestinal alkaline phosphatase (Qiagen cat. no 34510) which detects accessible histidine tail. The level of expression represents about 5% of total protein.

5. Coexpression With Thioredoxin

In an analagons fashion to the expression of prot D 1/3 E7 His from HPV 18 (example IX) an E. coli strain AR58 was transformed with a plasmid encoding thioredoxin and protein D 1/3 E7 His (HPV 16).

### EXAMPLE III

### Construction of an E. coli Strain Expressing Fusion Protein-D1/3-E6E7-his/HPV16

1. Construction of Expression Plasmid

a) Plasmid pMG MCS prot D1/3 (=pRIT14589) is a derivative of pMG81 (described Supra) in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p.119-125). The sequence of Prot-D1/3 is followed by a multiple cloning site (11 residues) and a coding region for a C-terminal histidine tail (6 His). This plasmid is used to express the fusion protein D1/3-E6E7-his.
b) HPV genomic E6 and E7 sequences type HPV16 (Seedorf et al., Virology 1985, 145, p.181-185) were amplified from HPV 16 full length genome cloned in pBR322 (obtained from Deutsches Krebsforschungszentrun (DKFZ), Referenzzentrum für human pathogen Papillomaviruses—D 69120—Heidelberg) and were subcloned into pUC19 to give TCA301 (=pRIT14462).
c) The coding sequences for E6 and E7 in TCA301 (=pRIT 14462) were modified with a synthetic oligonucleotides adaptor (inserted between Afl III and Nsi I sites) introducing a deletion of 5 nucleotides between E6 and E7 genes to remove the stop codon of E6 and create fused E6 and E7 coding sequences in the plasmid TCA309(=pRIT 14556).
   Construction of Plasmid TCA 311(=pRIT14512): a Plasmid Expressing the Fusion Protein-D1/3-E6E7-His/HPV16

The nucleotides sequences corresponding to amino acids 1→249 of fused E6E7 protein were amplified from pRIT14556. During the polymerase chain reaction, NcoI and SpeI restriction sites were generated at the 5′ and 3′ ends of the E6E7 fused sequences allowing insertion into the same sites of plasmid pMGMCS Prot D1/3 to give plasmid TCA311 (=pRIT14512). The insert was sequenced to verify that no modification had been generated during the polymerase chain reaction. The protein and coding sequence for the fusion protein-D E6/E7 1/3-His is described sequence ID No. 5 and 6.

2. Transformation of AR58 Strain

Plasmid pRIT14512 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of Prot-D1/3-E6E7-His

Cells of AR58 transformed with plasmid pRIT14512 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein D1/3-E6E7-his. The incubation at 39° C. was continued for 4 hours. Bacteria were pelleted and stored at −20C.

4. Characterization of Fusion Protein D1/3-E6E7-his

Frozen cells are thawed and resuspended in 10 ml of PBS buffer. Cells are broken in a French pressure cell press SLM Aminco at 20,000 psi (three passages). The extract is centrifuged at 16,000 g for 30 minutes at 4° C.

After centrifugation of extracts described above, aliquots of supernatant and pellet were analysed by SDS-polyacrylamide gel electrophoresis and Western blotting.

A major band of about 48 kDa, localized in the pellet fraction, was visualised by Coomassie stained gels and identified in Western blots by rabbit polyclonal anti-protein-D and by Ni-NTA conjugate coupled to calf intestinal alkaline phosphatase (Qiagen cat. no 34510) which detects accessible histidine tail. The level of expression represents about 1% of total protein.

### EXAMPLE: IV

In an analagous fashion the fusion protein of Lipo D 1/3 and E6-E7 from HPV16 was expressed in E. coli in the presence of thioredoxin.

The N-terminal of the pre-protein (388 aa) contains MDP residues followed by 16 amino acids of signal peptide of lipoprotein D (from Haemophilus Influenzae) which is cleaved in vivo to give the mature protein (370 aa). Lipoprotein portion (aa 1 to 127) is followed by the proteins E6 and E7 in fusion. The C terminal of the protein is elongated by TSGHHHHHH.

### EXAMPLE V

### Construction of E. coli Strain B1002 Expressing Fusion ProtD1/3-E7

Mutated (cys24->gly,glu26->gln) type HPV16

1)—Construction of Expression Plasmid

Starting Material:

a)—Plasmid pRIT 14501 (=TCA 308) which codes for fusion ProtD1/3-E7-His
b)—Plasmid LITMUS 28 (New England Biolabs cat no 306-28), a cloning vector pUC-derived
c)—Plasmid pMG MCS ProtD1/3 (pRIT 14589), a derivative of pMG81 (described Supra) in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p.119-125). The sequence of Prot-D1/3 is followed by a multiple cloning site (11 residues) and a coding region for a C-terminal histidine tail (6 His)
   Construction of Plasmid pRIT 14733(=TCA347): a Plasmid Expressing the Fusion Protein-D1/3-E7 Mutated (cys24->gly,glu26->gln) With His Tail

The NcoI-XbaI fragment from pRIT 14501 (=TCA 308), bearing the coding sequence of E7 gene from HPV16, elongated with an His tail, was subcloned in an intermediate vector Litmus 28 useful for mutagenesis to give pRIT 14909 (=TCA337) Double mutations cys24->gly (Edmonds and Vousden, J. Virology 63: 2650 (1989) and glu26->gln (Phelps et al, J. Virology 66: 2418-27 (1992) were chosen to impair the binding to the antioncogene product of Retinoblastome gene (pRB). The introduction of mutations in E7 gene was realized with the kit “Quick Change Site directed Mutagenesis (Stratagene cat n° 200518) to give plasmid pRIT 14681(=TCA343). After verification of presence of mutations and integrity of the complete E7 gene by sequencing, the mutated E7 gene was introduced into vector pRIT 14589 (=pMG MCS ProtD1/3) to give plasmid pRIT 14733 (=TCA347) protein and coding sequence.

The sequence for the fusion protein-D1/3-E 7 mutated (cys24->gly, glu26->gln)-His is described in sequence ID No. 7 and 8.

2)—Construction of Strain B1002 Expressing ProtD1/3-E7Mutated (cys 24->gly, glu26->gln)-His/HPV16

Plasmid pRIT 14733 was introduced into E. coli AR58 (Mott et al., 1985, Proc. Natl. Acad. Sci., 82:88) a defective λ lysogen containing a thermosensitive repressor of the λ pL promoter, to give strain B 1002, by selection for transformants resistant to kanamycine

3)—Growth and Induction of Bacterial Strain B1002—Expression of ProtD1/3-E7 Mutated (cys 24->gly, glu26->gln)-His/HPV16

Cells of AR58 transformed with plasmid pRIT 14733 (B1002 strain) were grown at 30° C. in 100 ml of LB medium supplemented with 50 μgr/ml of Kanamycin. During the logarithmic phase of growth bacteria were shifted to 39° C. to inactivate the λ repressor and turn on the synthesis of ProtD1/3-E7 mutated-His/HPV16. The incubation at 39° C. was continued for 4 hours. Bacteria were pelleted and stored at −20° C.

4)—Characterization of Fusion ProtD1/3-E7 mut (cys24->gly, glu26->gln)-His Type HPV16.

Frozen cells were thawed and resuspended in 10 ml of PBS buffer. Cells were broken in a French Pressure cell press SLM Aminco at 20 000 psi (three passages). The extract was centrifuged at 16000 g for 30 minutes at 4° C.

After centrifigation of extracts described above, aliquots of supernatant and pellet were analysed by SDS-polyacrylamide gel electrophoresis and Western blotting.

A major band of about 33 kDa, localized in the pellet fraction, was visualised by Coomassie stained gels and identified in Western blots by rabbit polyclonal 22 J 70 anti-protein D, by monoclonal anti E7/HPV16 from Zymed and by Ni-NTA conjugate coupled to calf intestinal alkaline phosphatase (Qiagen cat. no 34510) which detects accessible histidine tail. The level of expression represents about 3 to 5% of total protein.

Cells of B1002 were separated from the culture broth by centrifugation. The concentrated cells of B1002 were stored at −65° C.

### EXAMPLE VI

### Construction of an E. coli Strain Expressing Fusion clyta-E6-his (HPV 16)

1. Construction of Expression Plasmid

a)—Plasmid pRIT14497 (=TCA307), that codes for fusion ProtD1/3-E6-His/HPV16
b)—Plasmid pRIT14661 (=DVA2), an intermediate vector containing the coding sequence for the 117 C-terminal codons of LytA of Streptococcus Pneumoniae. Lyta is derived from Streptococcus pneumoniae which synthesize an N-acetyl-L-alanine amidase, amidase LYTA. (coded by the lytA gene {Gene, 43 (1986) pag 265-272} an autolysin that specifically degrades certain bonds in the peptidoglycan backbone. The C-terminal domain of the LYTA protein is responsible for the affinity to the choline or to some choline analogues such as DEAE.
   1.b Construction of Plasmid pRIT14634 (=TCA332): a Plasmid Expressing the Fusion clyta-E6-His/HPV16
a) The first step was the purification of the large NcoI-AflII restriction fragment from plasmid pRIT14497 and the purification of the small AflII-AflIII restriction fragment from pRIT14661
b) The second step was linking of clyta sequences to the E7-His sequences (NcoI and AflIII are compatible restriction sites) that gave rise to the plasmid pRIT 14634 (=TCA332), coding for the fusion protein clyta-E6-His under the control of the pL promoter.
   The protein and coding sequence for the fusion protein clyta-E6-His is described sequence ID No. 9 and 10.
   Transformation of AR58 Strain

Plasmid pRIT14634 was introduced into E. coli AR58 (Mott et al., 1985, Proc. Natl. Acad. Sci., 82:88) a defective λ lysogen containing a thermosensitive repressor of the λ pL promoter.

Growth and Induction of Bacterial Strain—Expression of clyta-E6-His

Cells of AR58 transformed with plasmid pRIT14634 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein clyta-E6-his. The incubation at 39° C. was continued for 4 hours. Bacteria were pelleted and stored at −20° C.

4. Characterization of Fusion clyta-E6-his

Frozen cells were thawed and resuspended in 10 ml of PBS buffer. Cells were broken in a French pressure cell press SLM Aminco at 20,000 psi (three passages). The extract was centrifuged at 16,000 g for 30 minutes at 4° C. After centrifugation of extracts described above, aliquots of supernatant and pellet were analysed by SDS-polyacrylamide gel electrophoresis and Western blotting.

A major band of about 33 kDa, localized in the pellet fraction, was visualised by Coomassie stained gels and identified in Western blots by rabbit polyclonal anti-clyta antibodies and by Ni-NTA conjugate coupled to calf intestinal alkaline phosphatase (Qiagen cat. no 34510) which detects accessible histidine tail. The level of expression represents about 3% of total protein.

### EXAMPLE VII

### Construction of an E. coli Strain Expressing Fusion clyta-E7-his (HPV 16)

1. Construction of Expression Plasmid

1.a Starting Materials

a)—Plasmid pRIT14501 (=TCA308), that codes for fusion ProtD1/3-E7-His/HPV16
b)—Plasmid pRIT14661 (=DVA2), an intermediate vector containing the coding sequence for the 117 C-terminal codons of LytA of Streptococcus Pneumoniae.
   1.b Construction of Plasmid pRIT14626 (=TCA330): a Plasmid Expressing the Fusion clyta-E7-His/HPV16
a) The first step was the purification of the large NcoI-AflII restriction fragment from plasmid pRIT14501 and the purification of the small AflII-AflIII restriction fragment from pRIT14661
b) The second step was linking of clyta sequences to the E7-His sequences (NcoI and AflIII are compatible restriction sites) that gave rise to the plasmid pRIT 14626 (=TCA330), coding for the fusion protein clyta-E7-His under the control of the pL promoter.

The protein and coding sequence for the fusion protein clyta-E7-His is decribed in sequence ID No. 11 and 12.

2. Transformation of AR58 Strain

Plasmid pRIT14626 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of clyta-E7-His

Cells of AR58 transformed with plasmid pRIT14626 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein clyta-E7-his. The incubation at 39° C. was continued for 4 hours. Bacteria were pelleted and stored at −20° C.

4. Characterization of Fusion clyta-E7-his

Frozen cells were thawed and resuspended in 10 ml of PBS buffer. Cells were broken in a French pressure cell press SLM Aminco at 20,000 psi (three passages). The extract was centrifuged at 16,000 g for 30 minutes at 4° C. After centrifugation of extracts described above, aliquots of supernatant and pellet were analysed by SDS-polyacrylamide gel electrophoresis and Western blotting.

A major band of about 35 kDa, localized in the pellet fraction, was visualised by Coomassie stained gels and identified in Western blots by rabbit polyclonal anti-clyta antibodies and by Ni-NTA conjugate coupled to calf intestinal alkaline phosphatase (Qiagen cat. no 34510) which detects accessible histidine tail. The level of expression represents about 5% of total protein.

### EXAMPLE VIII

### Construction of an E. coli Strain Expressing Fusion clyta-E6E7-his (HPV 16)

1. Construction of Expression Plasmid

1.a Starting Materials

a)—Plasmid pRIT14512 (=TCA311), that codes for fusion ProtD1/3-E6E7-His IHPV16
b)—Plasmid pRIT14661 (=DVA2), an intermediate vector containing the coding sequence for the 117 C-terminal codons of LytA of Streptococcus Pneumoniae.
   1.b Construction of Plasmid pRIT14629 (=TCA331): a Plasmid Expressing the Fusion clyta-E6E7-His/HPV16
a) The first step was the purification of the large NcoI-AflII restriction fragment from plasmid pRIT14512 and the purification of the small AflII-AflIII restriction fragment from pRIT14661
b) The second step was linking of clyta sequences to the E7-His sequences (NcoI and AflIII are compatible restriction sites) that gave rise to the plasmid pRIT 14629 (=TCA331), coding for the fusion protein clyta-E6E7-His under the control of the pL promoter.

The protein and coding sequence for the fusion protein clyta-E6E7-His is sequenced ID No. 13 and 14.

2. Transformation of AR58 Strain

Plasmid pRIT14629 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of clyta-E6E7-His

Cells of AR58 transformed with plasmid pRIT14629 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein clyta-E6E7-his. The incubation at 39° C. was continued for 4 hours. Bacteria were pelleted and stored at −20° C.

4. Characterization of Fusion clyta-E6E7-his

Frozen cells were thawed and resuspended in 10 ml of PBS buffer. Cells were broken in a French pressure cell press SLM Aminco at 20,000 psi (three passages). The extract was centrifuged at 16,000 g for 30 minutes at 4° C.

After centrifugation of extracts described above, aliquots of supernatant and pellet were analysed by SDS-polyacrylamide gel electrophoresis and Western blotting.

A major band of about 48 kDa, localized in the pellet fraction, was visualised by Coomassie stained gels and identified in Western blots by rabbit polyclonal anti-clyta antibodies and by Ni-NTA conjugate coupled to calf intestinal alkaline phosphatase (Qiagen cat. no 34510) which detects accessible histidine tail. The level of expression represents about 1% of total protein.

### EXAMPLE IX

### Prot D1/3 E7 his (HPV 18) (E. Coli B1011)

Protein D1/3 E7 his HPV Expressed With Thioredoxin in Trans (E. Coli B1012)

1)—Construction of Expression Plasmids

1).a. Construction of Plasmid TCA316(=pRIT 14532) a Plasmid Expressing the Fusion Protein-D1/3-E7-His/HPV18

Starting Materials

a)—Plasmid pMG MCS prot D1/3 (=pRIT14589) is a derivative of pMG81 (described in UK patent application no 951 3261.9 published as WO97/01640 in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991. Infection and Immunity, Jan. p.119-125). The sequence of Prot-D1/3 is followed by a multiple cloning site (11 residues) and a coding region for a C-terminal histidine tail (6 His). This plasmid is used to express the fusion protein D1/3-E7-his.
b)—HPV genomic E6 and E7 sequences of prototype HPV 18(Cole et al. J. Mol. Biol. (1987)193.599-608) were amplified from HPV16 full length genome cloned in pBR322 (obtained from Deutsche Krebsforschungszentrum (DKFZ), Referenzzentrum für human pathogen Papillomaviruses—D 69120—Heidelberg) and were subcloned into pUC19 to give TCA 302 (=pRIT14467).
   Construction of Plasmid TCA 316(=pRIT14532)

The nucleotides sequences corresponding to amino acids 1→105 of E7 protein were amplified from pRIT14467. During the polymerase chain reaction, NcoI and SpeI restriction sites were generated at the 5′ and 3′ ends of the E7 sequences allowing insertion into the same sites of plasmid pMGMCS Prot D1/3 to give plasmid TCA316 (=pRIT14532). The insert was sequenced and a modification versus E7HPV 18 prototype sequence was identified in E7 gene (nucleotide 128 G->A) generating a substitution of a glycine by a glutamic acid (aa 43 in E7, position 156 in fusion protein). The protein and coding sequence for the fusion protein-D1/3-E7-His/HPV18 is set forth in sequence ID No. 15 and ID No. 16.

1).b. Construction of Plasmid TCA313 (=pRIT14523): a Plasmid Expressing Thioredoxin

Starting Materials

a)—Plasmid pBBR1MCS4(Antoine R. and C. Locht, Mol. Microbiol. 1992,6,1785-1799; M. E. Kovach et al. Biotechniques 16, (5), 800-802) which is compatible with plasmids containing ColE1 or P15a origins of replication.
b)—Plasmid pMG42 (described in WO93/04175) containing the sequence of promoter pL of Lambda phage
c)—Plasmid pTRX (Invitrogen, kit Thiofusion K350-01) bearing the coding sequence for thioredoxin followed by AspA transcription terminator.
   Construction of Plasmid TCA313(=pRIT14523)

The fragment EcoRI-NdeI fragment from pMG42, bearing pL promoter and the NdeI-HindIII fragment from pTRX, bearing the coding sequence for thioredoxin followed by AspA terminator, were purified and ligated into the EcoRI and HindIII sites of plasmid vector pBBR1MCS4 to give plasmid TCA313(=pRIT14523).

The coding sequence for thioredoxin is described in ID No. 17.

2)—Transformation of AR58 Strain

2).a. To Obtain Strain B1011 Expressing ProtD1/3-E7-His/HPV18

Plasmid pRIT14532 was introduced into E. coli AR58 (Mott et al., 1985. Proc. Natl. Acad. Sci., 82:88) a defective λ lysogen containing a thermosensitive repressor of the λ pL promoter, by selection for transformants resistant to kanamycine.

2).b. Construction of Strain B1012 Expressing ProtD1/3-E7-His/HPV18 and Thioredoxin

Plasmid pRIT14532 and pRIT14523 were introduced into E. coli AR58 (Mott et al., 1985, Proc. Natl. Acad. Sci., 82:88) a defective λ lysogen containing a thermosensitive repressor of the λ pL promoter, by double selection for transformants resistant to kanamycin and ampicillin.

3)—Growth and Induction of Bacterial Strains B1011 and B1012—Expression of Prot-D1/3-E7-His/HPV18 Without and With Thioredoxin in Trans

Cells of AR58 transformed with plasmids pRIT14532 (B1011 strain) and Cells of AR58 transformed with plasmids pRIT14532 and pRIT14523 (B1012 strain) were grown at 30° C. in 100 ml of LB medium supplemented with 50 μgr/ml of Kanamycin for B1011 strain and supplemented 50 μgr/ml of Kanamycin and 100 μgr/ml of Ampicillin for B1012 strain. During the logarithmic phase of growth bacteria were shifted to 39° C. to inactivate the 1 repressor and turn on the synthesis of protein D1/3-E7-his/HPV18 and thioredoxin. The incubation at 39° C. was continued for 4 hours.

Characterization of Fusion Protein D1/3-E7-his/HPV18

Preparation of Extracts

Frozen cells are thawed and resuspended in 10 ml of PBS buffer. Cells are broken in a French pressure cell press SLM Aminco at 20,000 psi (three passages). The extract is centrifuged at 16,000 g for 30 minutes at 4° C.

Analysis on Coomassie-Stained SDS-Polyacrylamide Gels and Western Blots

After centrifugation of extracts described above, aliquots of supernatant and pellet were analysed by SDS-polyacrylamide gel electrophoresis and Western blotting.

The fusion protD1/3-E7-His (about 31 kDa) was visualised by Coomassie stained gels in the pellet fraction for strain B1011 and partially localized (30%) in the supernatant fraction for strain B1012 and was identified in Western blots by rabbit polyclonal anti-protein-D and by Ni-NTA conjugate coupled to calf intestinal alkaline phosphatase (Qiagen cat. no 34510) which detects accessible histidine tail. The level of expression represents about 1-3% of total protein as shown on a Coomassie-stained SDS-polyacrylamide gel.

For the extract of strain B1012 the thioredoxin (about 12 KDa) was visualised by coomassie stained gel in the supernatant and identified in western blots by monoclonal anti thioredoxin (Invitrogen R920-25)

### EXAMPLE X

### Construction of E. coli Strain B1098 Expressing Fusion ProtD1/3-E7

Mutated (cys27->gly,glu29->gln) Type HPV18

1)—Construction of Expression Plasmid

Starting Material:

a)—Plasmid pRIT 14532 (=TCA 316) which codes for fusion ProtD1/3-E7-His
b)—Plasmid LITMUS 28 (New England Biolabs cat n° 306-28), a cloning vector pUC-derived
c)—Plasmid pMG MCS ProtD1/3 (pRIT 14589), a derivative of pMG81 (described supra) in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p.119-125). The sequence of Prot-D1/3 is followed by a multiple cloning site (11 residues) and a coding region for a C-terminal histidine tail (6 His)
   Construction of Plasmid pRIT 14831(=TCA355): a Plasmid Expressing the Fusion Protein-D1/3-E7 Mutated (cys27->gly,glu29->gln) With His Tail

The NcoI-XbaI fragment from pRIT 14532 (=TCA 316), bearing the coding sequence of E7 gene from HPV 18, elongated with an His tail, was subcloned in an intermediate vector Litmus 28 useful for mutagenesis to give pRIT 14910 (=TCA348) By analogy with E7/HPV16 mutagenesis, double mutations cys27->gly and glu29->gln were chosen to impair the binding to the antioncogene product of Retinoblastome gene (pRB).

The introduction of mutations in E7 gene was realized with the kit “Quick Change Site directed Mutagenesis (Stratagene cat no 200518). As the sequencing of pRIT14532 had pointed out the presence of a glutamic acid in position 43 of E7 instead of a glycine in the prototype sequence of HPV 18, a second cycle of mutagenesis was realized to introduce a glycine in position 43. We obtained plasmid pRIT 14829 (=TCA353). After verification of presence of mutations and integrity of the complete E7 gene by sequencing, the mutated E7 gene was introduced into vector pRIT 14589 (=pMG MCS ProtD1/3) to give plasmid pRIT 14831 (=TCA355).

The protein and coding sequence for the fusion protein-D1/3-E 7 mutated (cys27->gly, glu29->gln)-His is described in sequence ID No. 18 and 19.

2) Construction of Strain B1098 Expressing ProtD1/3-E7Mutated (cys 27->gly, glu29->gln)-His/HPV18

Plasmid pRIT 14831 was introduced into E. coli AR58 (Mott et al., 1985, Proc. Natl. Acad. Sci., 82:88) a defective λ lysogen containing a thermosensitive repressor of the λ pL promoter, to give strain B1098, by selection for transformants resistant to kanamycin.

3)—Growth and Induction of Bacterial Strain B1098—Expression of ProtD1/3-E7 Mutated (cys 27->gly, glu29->gln)-His/HPV18

Cells of AR58 transformed with plasmid pRIT 14831 (B1098 strain) were grown at 30° C. in 100 ml of LB medium supplemented with 50 μgr/ml of Kanamycin. During the logarithmic phase of growth bacteria were shifted to 39° C. to inactivate the λ repressor and turn on the synthesis of ProtD1/3-E7 mutated-His/HPV18. The incubation at 39° C. was continued for 4 hours. Bacteria were pelleted and stored at −20° C.

4) Characterization of Fusion ProtD1/3-E7 mut (cys24->gly, glu26->gln)—His Type HPV16

Frozen cells were thawed and resuspended in 10 ml of PBS buffer. Cells were broken in a French Pressure cell press SLM Aminco at 20 000 psi (three passages). The extract was centrifuged at 16000 g for 30 minutes at 4° C.

Analysis on Coomassie Stained SDS-Polyacrylamide Gels and Western Blots

After centrifugation of extracts described above, aliquots of supernatant and pellet were analysed by SDS-polyacrylamide gel electrophoresis and Western blotting. A major band of about 31 kDa, localized in the pellet fraction, was visualised by Coomassie stained gels and identified in Western blots by rabbit polyclonal 22 J 70 anti-protein D and by monoclonal Penta-His (Qiagen cat. no 34660) which detects accessible histidine tail. The level of expression represents about 3 to 5% of total protein.

### EXAMPLE XI

### Construction of an E. coli Strain Expressing Fusion Protein-D1/3-E6-his/HPV18

1. Construction of Expression Plasmid

a) Plasmid pMG MCS prot D1/3 (=pRIT14589) is a derivative of pMG81 (described supra) in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p.119-125). The sequence of Prot-D1/3 is followed by a multiple cloning site (11 residues) and a coding region for a C-terminal histidine tail (6 His). This plasmid is used to express the fusion protein D1/3-E6-his.
   HPV genomic E6 and E7 sequences type HPV18 (Cole et al., J. Mol. Biol. 1987, 193, p.599-608.) were amplified from HPV18 full length genome cloned in pBR322 (obtained from Deutsches Krebsforschungszentrum (DKFZ), Referenzzentrurn für human pathogen Papillomaviruses—D 69120—Heidelberg) and were subcloned into pUC19 to give TCA 302 (=pRIT14467).
   Construction of Plasmid TCA 314(=pRIT14526): a Plasmid Expressing the Fusion Protein-D1/3-E6-His/HPV18

The nucleotides sequences corresponding to amino acids

1→158 of E6 protein were amplified from pRIT14467. During the polymerase chain reaction. NcoI and SpeI restriction sites were generated at the 5′ and 3′ ends of the E6 sequences allowing insertion into the same sites of plasmid pMGMCS Prot D1/3 to give plasmid TCA314 (=pRIT14526). The insert was sequenced to verify that no modification had been generated during the polymerase chain reaction. The protein and coding sequence for the fusion protein-D1/3-E6-His is described in sequence ID No. 20 and 21.

Transformation of AR58 Strain

Plasmid pRIT14526 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of Prot-D1/3-E6-His

Cells of AR58 transformed with plasmid pRIT14526 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein D1/3-E6-his. The incubation at 39° C. was continued for 4 hours. Bacteria were pelleted and stored at −20C.

4. Characterization of Fusion Protein D1/3-E6-his

Frozen cells are thawed and resuspended in 10 ml of PBS buffer. Cells are broken in a French pressure cell press SLM Aminco at 20,000 psi (three passages). The extract is centrifuged at 16,000 g for 30 minutes at 4° C. After centrifugation of extracts described above, aliquots of supernatant and pellet were analysed by SDS-polyacrylamide gel electrophoresis and Western blotting.

A major band of about 32 kDa, localized in the pellet fraction, was visualised by Coomassie stained gels and identified in Western blots by rabbit polyclonal anti-protein-D and by Ni-NTA conjugate coupled to calf intestinal alkaline phosphatase (Qiagen cat. no 34510) which detects accessible histidine tail. The level of expression represents about 3-5% of total protein.

### EXAMPLE XII

### Construction of an E. coli Strain Expressing Fusion Protein-D1/3-E6E7-his/HPV18

1. Construction of Expression Plasmid

a) Plasmid pMG MCS prot D1/3 (=pRIT14589) is a derivative of pMG81 (described supra) in which the codons 4-81 of NS1 coding region from Influenza were replaced by the codons corresponding to residues Ser 20→Thr 127 of mature protein D of Haemophilus Influenzae strain 772, biotype 2 (H. Janson et al., 1991, Infection and Immunity, Jan. p.119-125). The sequence of Prot-D1/3 is followed by a multiple cloning site (11 residues) and a coding region for a C-terminal histidine tail (6 His). This plasmid is used to express the fusion protein D1/3-E6E7-his.
b) HPV genomic E6 and E7 sequences type HPV18 (Cole et al. J. Mol. Biol. 1987, 193, 599-608) were amplified from HPV18 full length genome cloned in pBR322 (obtained from Deutsches Krebsforschungszentrum (DKFZ), Referenzzentrum fir human pathogen Papillomaviruses—D 69120—Heidelberg) and were subcloned into pUC19 to give TCA 302 (=pRIT14467).
c) The coding sequences for E6 and E7 in TCA302 (=pRIT 14467) were modified with a synthetic oligonucleotides adaptor (inserted between Hga I and Nsi I sites) introducing a deletion of 11 nucleotides between E6 and E7 genes, removing the stop codon of E6 and creating fused E6 and E7 coding sequences in the plasmid TCA320(=pRIT 14618).
   Construction of Plasmid TCA 328(=pRIT14567): a Plasmid Expressing the Fusion Protein-D1/3-E6E7-His/HPV18

The nucleotides sequences corresponding to amino acids

1 →263 of fused E6E7 protein were amplified from pRIT14618. During the polymerase chain reaction, NcoI and SpeI restriction sites were generated at the 5′ and 3′ ends of the E6E7 fused sequences allowing insertion into the same sites of plasmid pMGMCS Prot D1/3 to give plasmid TCA328 (=pRIT14567). The insert was sequenced to verify that no modification had been generated during the polymerase chain reaction. The protein and coding sequence for the fusion protein-D1/3-E6E7-His is described in sequence ID No. 22 and 23.

2. Transformation of AR58 Strain

Plasmid pRIT14567 was introduced into E. coli AR58 (Mott et al., 1985, Proc. Natl. Acad. Sci., 82:88) a defective λ lysogen containing a thermosensitive repressor of the λ pL promoter.

3. Growth and Induction of Bacterial Strain—Expression of Prot-D1/3-E6E7-His

Cells of AR58 transformed with plasmid pRIT14512 were grown in 100 ml of LB medium supplemented with 50 μgr/ml of Kanamycin at 30° C. During the logarithmic phase of growth bacteria were shifted to 39° C. to inactivate the λ repressor and turn on the synthesis of protein D1/3-E6E7-his. The incubation at 39° C. was continued for 4 hours. Bacteria were pelleted and stored at −20C.

4. Characterization of Fusion Protein D1/3-E6E7-his

Frozen cells are thawed and resuspended in 10 ml of PBS buffer. Cells are broken in a French pressure cell press SLM Aminco at 20,000 psi (three passages). The extract is centrifuged at 16,000 g for 30 minutes at 4° C.

After centrifugation of extracts described above, aliquots of supernatant and pellet were analysed by SDS-polyacrylamide gel electrophoresis and Western blotting.

A major band of about 48 kDa, localized in the pellet fraction, was visualised by Coomassie stained gels and identified in Western blots by rabbit polyclonal anti-protein-D and by Ni-NTA conjugate coupled to calf intestinal alkaline phosphatase (Qiagen cat. no 34510) which detects accessible histidine tail. The level of expression represents about 1% of total protein.

### EXAMPLE XIII

The therapeutic potential of vaccine containing the PD1/3 E7 fusion protein and different CpG oligonucleotides were evaluated in the TC1 (E7 expressing tumour model.)

1. Therapeutic Experiments: Protocol

10e6 TC1 cells, E7 expressing tumour cells: were injected subcutaneously (200 μl) in the flank of C57BL/6 immunocompetent mice. Mice were vaccinated 7 and 14 days after the tumour challenge, with 5 μg ProtD 1/3 E7 HPV 16 injected intra-footpad (100 μl: 50 μl/footpad) in the presence of different adjuvants:

2 and 4 weeks after the second immunisation, 5 mice/group were killed and spleens or popliteal lymph nodes were taken and analyzed for immune response.

1.2 Results

Groups of Mice
1) PBS
2) ProtD1/3 E7 HPV16
3) ProtD1/3 E7 HPV16+oligo 1: 1826 (WD 1001): TCC ATG ACG TTC CTG ACG TT
4) Oligo 1
5) ProtD1/3 E7 HPV16+oligo 2/1758 (WD1002): TCT CCC AGC GTG CGC CAT
6) Oligo 2

Tumour Growth;
was monitored by measuring individual tumours twice a week.

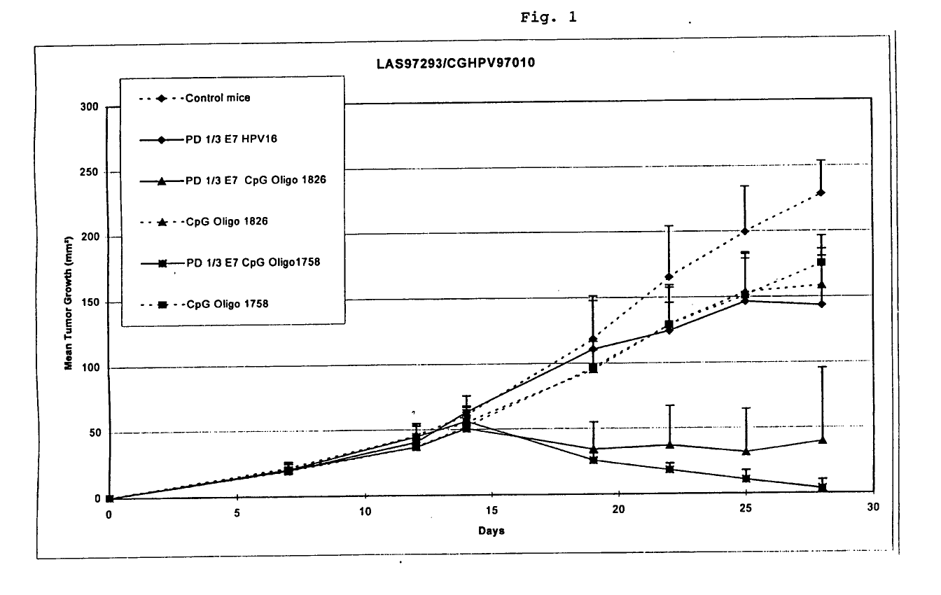

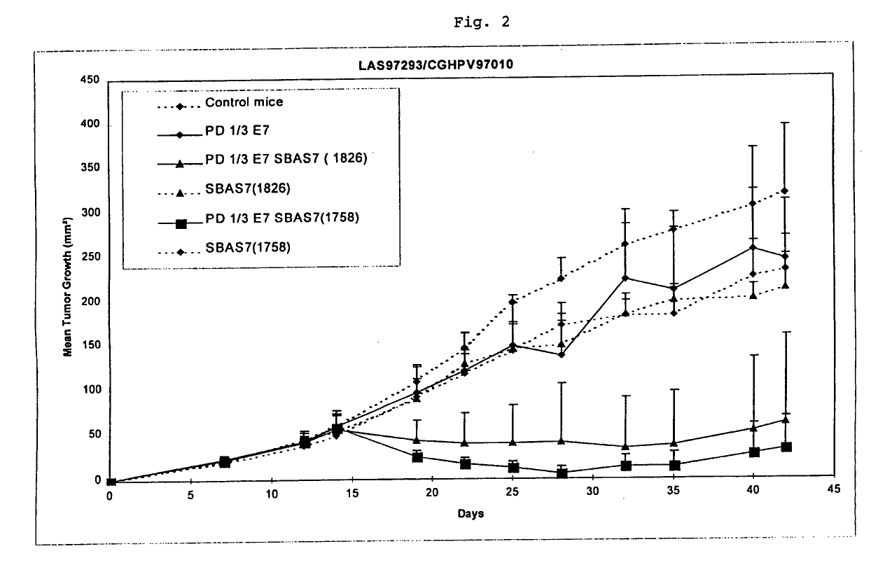

FIG. 1: represents the mean tumour growth (in mm2)/group n=10 followed over 4 weeks.
The injection of 10e6 TC1 cells injected subcutaneously give rise to a growing tumour in 100% of the animals.
Vaccinating with ProtD1/3E7 or adjuvant alone: 100% of the animals develop a tumour.
As shown in FIGS. 1 and 2, in the groups of mice that received the antigen with a CpG oligonucleotide the mean tumour growth remained very low and very similar between groups, reflecting that the tumour growth either was slowed down or that several tumours were completely rejected.

The analysis of individual tumour growth 2 and 4 weeks after the latest vaccination showed that complete rejection in the groups were:

| | Day 28 (n = 10) | day 42 (n = 5) |
|---|---|---|
| E7 + oligo1 (1826) | 40% | 40% |
| Oligo1 | 0% | 0% |
| E7 + oligo2 (1758) | 70% | 40% |
| Oligo2 | 0% | 0% |

The mean tumour growth/group of mice vaccinated with PD1/3 E7+ the CpG oligos are quite similar and analysis of the individual tumour growth showed that the CpG oligos induce prolonged complete tumour rejection.

Conclusion

Both CpG (Oligo 2>oligo 1) induced complete tumour regression.

Lymphoproliferative response was analysed by in vitro restimulation of spleen and lymph nodes cells for 72 hrs with either PD1/3E7, the protein E7(Bollen) and PD (whole) PD1/3 (coated or not on latex μbeads) (10, 1, 0.1 μg/ml) 2 and 4 weeks post II.
Positive controls (ConA stimulaltion) were positive.
Surprisingly, no E7 specific and no PD specific proliferative response could be observed starting with spleen cells 2 or 4 weeks post II (probably due to a technical problem: data not shown).
On the contrary, lymph node cells from mice that received ProtD1/3 E7 in CpG oligos 1 and 2 showed a very good E7 specific proliferative response although almost no PD (whole) specific response could be observed even at the hightest concentration of 100 μg/ml no PD1/3 specific responses was observed even when coated on latex μbeads.

Similar data were obtained 4 weeks post II.

Serology

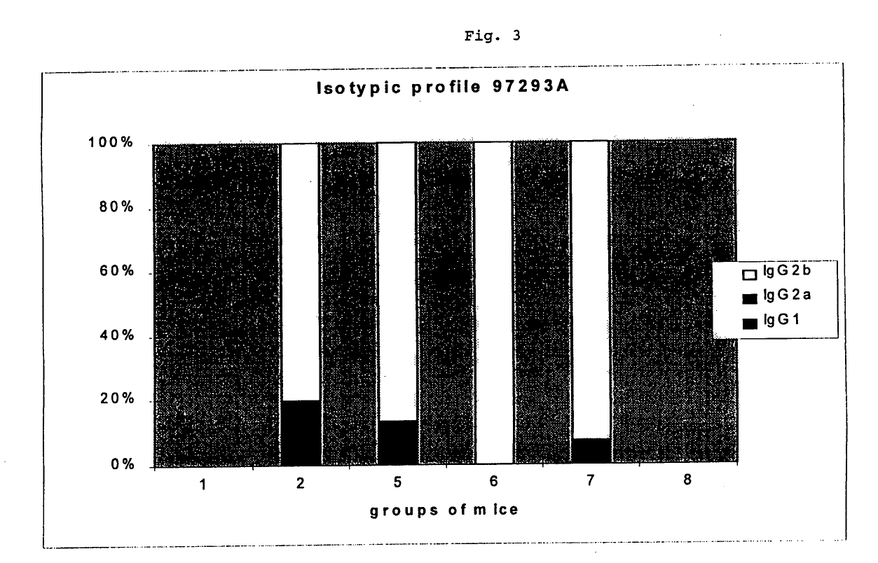

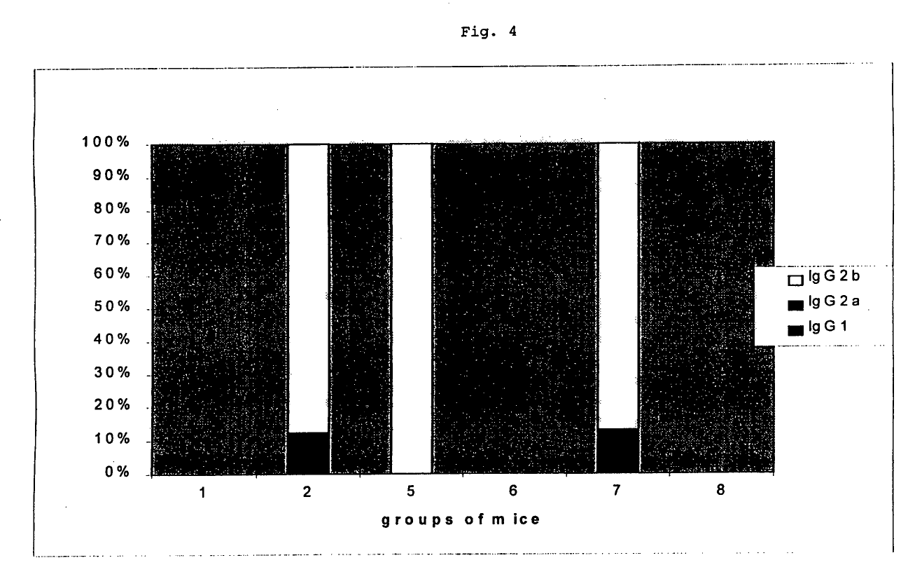

The anti E7 antibody response: IgG tot and isotypes (IgG1, IgG2a, IgG2b, IgGTot) were measured by ELISA using the E7 protein as coating antigen as described in the Materials and Methods. FIGS. 3 and 4 show the relative percentage of the different IgG isotypes in the total of IgGs, 2 and 4 weeks post II respectively.
The Oligos affect only weakly (oligo 2) or not at all (Oligo 1) the weak antibody response observed when PD1/3E7 alone was injected.
The predominant E7 specific antibody subclass was clearly IgG2b for all the formulation tested (80-90% of the total IgGs).

The same results were obtained 4 weeks post II

Isotypic Profile of Anti E7 Responses (Post II, Pooled Sera) Exp. 97293

| Groups | IgG1 | IgG2a | IgG2b | IgGtot |
|---|---|---|---|---|
| 1) PBS | 0 | 0 | 0 | 0 |
| 2) ProtD1/3 E7 HPV16 | 1020 | 0 | 4130 | 4740 |
| 3) ProtD1/3 E7 HPV16 + oligo 1 | 170 | 400 | 3680 | 4910 |
| 4) Oligo 1 | 0 | 0 | 530 | 420 |
| 5) ProtD1/3 E7 HPV16 + oligo 2 | 0 | 590 | 7560 | 13690 |
| 6) Oligo 2 | 0 | 0 | 0 | 0 |
| 1) PBS | 0 | 0 | 0 | 0 |
| 2) ProtD1/3 E7 HPV1 | 240 | 0 | 1650 | 1400 |
| 3) ProtD1/3 E7 HPV16 + oligo 1 | 0 | 0 | 1280 | 1430 |
| 4) Oligo 1 | 0 | 0 | 0 | 0 |
| 5) ProtD1/3 E7 HPV16 + oligo 2 | 0 | 560 | 3600 | 5880 |
| 6) Oligo 2 | 0 | 0 | 0 | 0 |

CTL Assay:

A CTL response could be detected when measured 2 weeks after the latest vaccination, when cells were re-stimulated in vitro with irradiated TC1 when TC1 or peptide E7 pulsed EL4, were used as target cells, when mice immunised with PD1/3 E7+CpG oligo 2>1 (25-40% specific lysis) and not with oligos alone.
Lysis was seen on TC1 cells than on peptide E7 pulsed EL4 cells, but this is mostly observed in the groups of mice vaccinated with PD1/3E7+CpG oligos (2>1). In this experiment other formulations did not induce a CTL.
Using E7 pulsed EL4 cells, no lysis was observed when mice received the protein or the adjuvant alone.

1.3 Materials and Methods

| | | | Concentration | |
|---|---|---|---|---|
| Component | Brand | Batch number | (mg/ml) | Buffer |
| ProtD1/3-E7 | | 957/015 | 0.677 | PBS 7.4 |
| oligo CpG | EuroGentec | WD1001 | 5 | H₂0 |
| 1826 | | | | |
| oligo CpG | EuroGentec | WD1002 | 5 | H₂0 |

1.3.1 Formulation Process

All the formulations were prepared on the day of injection.

Oligo Containing Formulations

Formulations containing oligo alone without other adjuvant were prepared by addition of CpG to the diluted PrtD1/3-E7 in PBS pH 7.4.

The adjuvant controls without antigen were prepared by replacing the protein by PBS.

1.3.2 Mice and Cell Lines

Mice C57B1/6 (Iffa Credo) 6-8 weeks old mice were used in these experiments.

Cell lines: TC1 (obtained from the John Hopkin's University), or EL4 cells were grown in RPMI 1640 (Bio Whittaker) containig 10% FCS and additives: 2 mM L-Glutamine. 1% antibiotics (10000 U/ml penicilin. 10000 μg/ml streptomycin) 1% non essential amino acid 100×, 1% sodium pyruvate (Gibco), 5 10e-5 M 2-mercaptoethanol. Before injection TC1 cells were trypsynized and washed in serum free medium.

1.3.3 Tumour Growth:

All the animals were injected with tumor cells on day 0 and were randomized at day 7. Individual tumor growth was followed over time (the 2 main diameters (A, B) were measured using calipers twice a week, A×B represents the “tumor surface” and the average of the 5 values/groups is showed on a graphic over time: 6 weeks

1.3.4 CMI Read Out

In Vitro Lymphoproliferation

Lymphoproliferation was performed on individual spleens and on lymph node pools. 200000 spleen cells or popliteal lymph node cells were plated in triplicate, in 96 well microplate, in RPMI medium containing 1% normal mice serum and additives. After 72 hrs of in vitro re-stimulation with different amounts of PD1/3 E7 (1, 0.1, 0.01 μl/ml) or E7 (10-1-0.1 μg/ml) After 72 hrs, 100 μl of culture supernatant were removed and replaced by fresh medium containing 1 μCi 3H thymidine (Amersham 5 Ci/mmol). After 16 hrs, cells were harvested onto filter plates. Incorporated radioactivity was counted in a β counter. Results are expressed in CPM (mean of triplicate wells) or as stimulation indexes (mean CPM in cultures with antigen/mean CPM in cultures without antigen).

1.3.5 CTL Assay

20 10e6 spleen cells were co-cultured with 2 10e6 irradiated (18000 r) TC1 cells (E7 expressing tumor) for 7 days in the presenced or absence of ConA sup. (2%)

Target cells used to assess cytotoxicity were either Cr51 (DuPont NEN 37 MBq/ml) loaded (1 hr at 37° C.) TC1 cells or E7 pulsed EL4 cells (for 1 hr at 37° C. during the Cr 51 loading of the cells 10 μg/ml of E7-derived peptide (49-57) (QCB) compared to EL4 cells NK dependant lysis was assessed on K562 target cells 2000 target cells were added/well of 96 well plate (V bomom nunc 2-45128) with 100/1 being the highest Effector/target ratio. Controls for spontaneous or maximal Cr51 release were performed in sextuplet and were targets in medium or in triton 1.5%. All plates were gently centrifuged and incubated for 4 hrs at 37 in 7% CO2. 50 μl of the supernatant was deposed on 96 w Lunaplate (Packard) let dry O/N and counted in a Top Count counter. Data is expressed as percent specific lysis which is calculated from the c.p.m. by the formula (experimental release−spontaneous release)/(maximal release−spontaneous release)×100.

Serology

Quantitation of anti E7 antibody was performed by Elisa using E7 as coating antigen. Antigen and antibody solutions were used at 50 μl per well. Antigen was diluted at a final concentration of 3 μg/ml in carbonate buffer ph9.5 and was adsorbed overnight at 4° C. to the wells of 96 wells microtiter plates (Maxisorb Immuno-plate, Nunc, Denmark). The plates were then incubated for 1 hr at 37° C. with PBS containing 1% bovine serum albumin and 0.1% Tween 20 (saturation buffer). Two-fold dilutions of sera (starting at 1/100 dilution) in the saturation buffer were added to the E7-coated plates and incubated for 1 hr 30 min at 37° C. The plates were washed 3 times with PBS 0.1% Tween 20 and biotin-conjugated anti-mouse IgG1, IgG2a or IgG2b or IgGtot (Amersham, UK) diluted 1/5000 in saturation buffer was added to each well and incubated for 1 hr 30 min at 37° C. After a washing step, streptavidin-biotinylated peroxydase complex (Amersham, UK) diluted 1/5000 in saturation buffer was added for an additional 30 min at 37° C. Plates were washed as above and incubated for 10 min with TMB (tetra-methyl-benzidine). The reaction was stopped with H2SO4 4N and read at 450 nm. Midpoint dilutions were calculated by SoftmaxPro (using a four parameters equation).

### EXAMPLE XIV

In a second experiment, the vaccine of the invention were tested to assess the significance of the backbone:

Therapeutic Experiment: Protocol

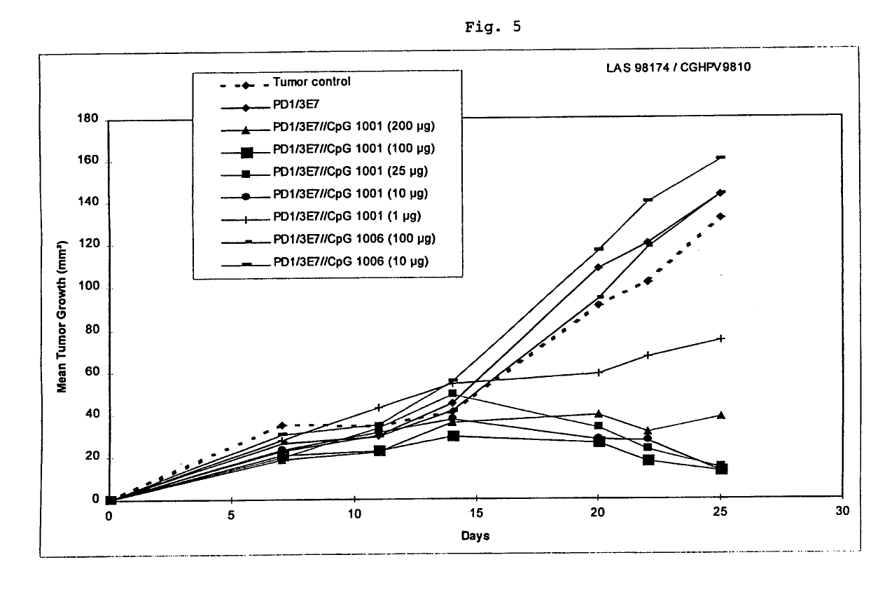

10e6 TC1 cells, E7 expressing tumor cells: were injected subcutaneously (200 μl) in the flank of immunocompetent C57BL/6 mice.
2 vaccinations, 7 and 14 days after the tumor challenge, with 5>g ProtD 1/3 E7 HPV16 injected intra-footpad (100 μl: 50 μl/footpad)+/−CpG oligo; Oligo 1 (WD1001) as a phosphorothioate modified or the same Oligo (WD1006) but with phosphodiester linkage.
5 animals/group.
   The tumor growth was monitored by measuring individual tumors twice a week and the mean tumor growth/group of 5 animals is depicted in FIG. 5 and show the phosphorothioate modified oligonucleotides are effective in bringing about tumour regression.
   Conclusions:
All the animals that received 10e6 TC1 tumor cells develop a growing tumor.
100% of the animals vaccinated twice, 7 days apart, with the PD1/3 E7 HPV16 protein alone develop a tumor.
100% of the animals receiving the PD1/3 E7 protein+oligo WD1006 develop a tumor at the concentrations tested
All the groups of animals that received the E7 protein+CpG 1001 at a concentration ranging from 10 to 200 μg show tumor regression partial or complete (20-40%).
   The first concentration at which this therapeutic effect on tumor regression is not fully obtained is E7+1 μg CpG oligo 1001.

### EXAMPLE XV

In a third series of experiments, the vaccines of the invention were evaluated in transgenic mice expressing E7 protein.

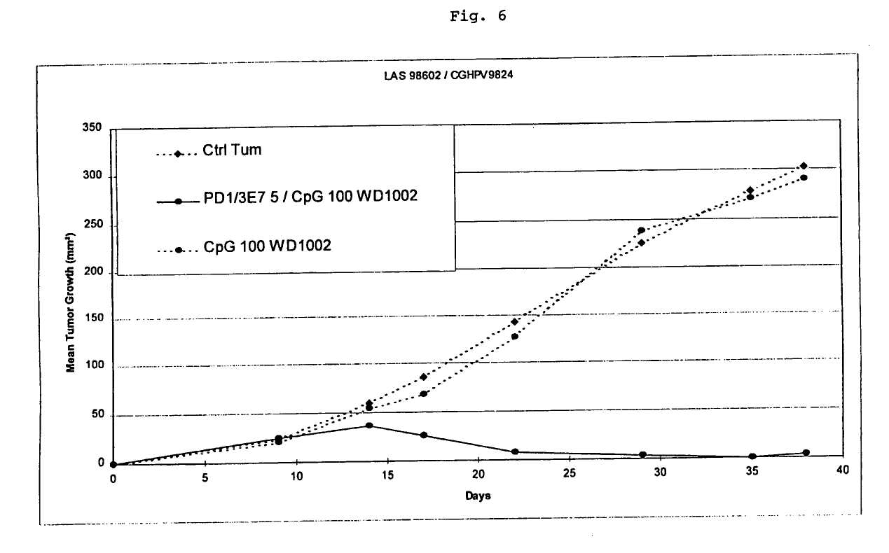

The transgenic mouse strain has been generated by M. Parmentier and C. Ledent at the IRIBHN (ULB). (Ref: PNAS (USA) 1990, 87; 6176-6180).
As transgenic mice live with the E7 HPV16 gene from birth, they are considered “tolerant” to this gene: E7 from HPV 16, in this situation is considered as a “self antigen”.
The expression of the transgene is driven by the thyroglobulin promoter. As Thyroglobulin is constitutively expressed only In the Thyroid, E7 is expressed in the thyroid.
As a consequence of this expression, thyroid cells proliferate, mouse develop goiter and nodules which after 6 months to 1 year can evoluate in invasive cancer.
   The results (FIG. 6) of the experiments show that therapeutic vaccination with CpG oligonucleotide and antigen as described herein, results in a reduction of tumour growth and can induce complete tumour regression.
   Material & Methods
10e6 TC1 cells, E7 expressing tumor cells: were injected subcutaneously (200 μl) in the flank of male or female C57BL/6 Transgenic
mice were vaccinated 7 and 14 days after the tumor challenge, with 51 g ProtD 1/3 E7 HPV16 injected intra-footpad (100 μl: 50 μl/footpad) in the 2 presence of CpG oligonucleotide TCT CCC AGC GTG CGC CAT and two control adjuvants,
10 animals/group
   2 and 4 weeks after the second immunization were killed and spleens or popliteal lymph.

### CONCLUSION

The vaccines of the invention are effective in bringing about tumour regression in HPV induced tumours.

1. A composition comprising an E6 or E7 protein or E6/E7 fusion protein from HPV optionally linked to an immunological fusion partner, and an immunomodulatory CpG oligonucleotide.

2. A composition as claimed in claim 1 wherein the fusion partner is selected from the group; protein D or a fragment thereof from Heamophilus influenzae B, lipoprotein D or fragment thereof from Heamophilus influenzae B, NS1 or fragment thereof from Influenzae Virus, and LYTA or fragment thereof from Streptococcus Pneumoniae.

3. A composition as claimed in claim 1 wherein the E6 or E7 proteins are derived from HPV16 or HPV18.

4. A composition as claimed in claim 1 wherein the E7 protein is mutated.

5. A composition as claimed in claim 1 wherein the E6 protein is mutated.

6. A composition as claimed in claim 1 additionally comprising a hisitidine tag of at least 4 hisitidine residues.

7. A composition as claimed herein comprising an additional HPV antigen.

8. A composition as claimed herein where the immumodulatory CpG oligonucleotide comprises a hexamer motif: purine purine cytosine guaine pyrimidine pyrimidine.

9. A composition as claimed herein wherein the immunomodulatory CpG oligonucleotide has two or more CpG motifs.

10. A composition as claimed herein wherein the CpG oligonucleotide contains a phosphorothioate inter-nucleotide linkage.

11. A composition as claimed herein wherein the CpG oligonucleotide is selected from the group:

| | |
|---|---|
| OLIGO 1: | TCC ATG ACG TTC CTG ACG TT |
| OLIGO 2: | TCT CCC AGC GTG CGC CAT |
| OLIGO 3: | ACC GAT GAC GTC GCC GGT GAC GGC ACC ACG |

12. A composition as claimed herein for use in medicine.

13. A method of inducing an immune response in a patient to an HPV antigen comprising administering a safe and effective amount of a composition as claimed herein.

14. A method of preventing or treating HPV induced tumours in a patient comprising administering a safe and effective amount of a composition as claimed herein.

15. A method of preparing a composition as claimed herein, comprising admixing an E6, E7 or E6/E7 fusion protein optionally linked to an immunological fusion partner, and an immunomodulatory CpG oligonucleotide.


SEQUENCE LISTING (28 sequences)

SEQ ID NO: 1 - protein, 220 aa, Artificial Sequence
  Chimaeric protein (protein D from Haemophilius influenzae B and E7 from Human papilloma virus type 16)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPTSGHHHHHH

SEQ ID NO: 2 - DNA, 663 bp, Artificial Sequence
  Chimaeric protein (protein D from Haemophilius influenzae B and E7 from Human papilloma virus type 16)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaactagtggccaccatcaccatcaccattaa

SEQ ID NO: 3 - DNA, 822 bp, Artificial Sequence
  Chimaeric protein (protein D from Haemophilius influenzae B and E6 from Human papilloma virus type 16)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgactagtggccaccatcaccatcaccattaa

SEQ ID NO: 4 - protein, 273 aa, Artificial Sequence
  Chimaeric protein (protein D from Haemophilius influenzae B and E6 from Human papilloma virus type 16)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLTSGHHHHHH

SEQ ID NO: 5 - DNA, 1116 bp, Artificial Sequence
  Chimaeric protein (protein D from Haemophilius influenzae B and E6E7 fusion from Human papilloma virus type 16)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaactagtggccaccatcaccatcaccattaa

SEQ ID NO: 6 - protein, 371 aa, Artificial Sequence
  Chimaeric protein (protein D from Haemophilius influenzae B and E6E7 fusion from Human papilloma virus type 16)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPTSGHHHHHH

SEQ ID NO: 7 - DNA, 663 bp, Artificial Sequence
  Chimaeric protein (protein D from Haemophilius influenzae B and mutated E7 from Human papilloma virus type 16)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgcatggagatacacctacattgcatgaatatatgttagatttgcaaccagagacaactgatctctacggttatcagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaactagtggccaccatcaccatcaccattaa

SEQ ID NO: 8 - protein, 220 aa, Artificial Sequence
  Chimaeric protein (protein D from Haemophilius influenzae B and mutated E7 from Human papilloma virus type 16)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMHGDTPTLHEYMLDLQPETTDLYGYQQLNDSSEEEDEIDGPAGQAEPDRAHYNIVTFCCKCDSTLRLCVQSTHVDIRTLEDLLMGTLGIVCPICSQKPTSGHHHHHH

SEQ ID NO: 9 - DNA, 879 bp, Artificial Sequence
  Chimaeric protein (Clyta from Streptococcus pneumoniae and E6 from Human papilloma virus type 16)
atgaaagggggaattgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattggccagcatgctggacatggccatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgactagtggccaccatcaccatcaccattaa

SEQ ID NO: 10 - protein, 292 aa, Artificial Sequence
  Chimaeric protein (Clyta from Streptococcus pneumoniae and E6 from Human papilloma virus type 16)
MKGGIVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPELASMLDMAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLTSGHHHHHH

SEQ ID NO: 11 - DNA, 720 bp, Artificial Sequence
  Chimaeric protein (Clyta from Streptococcus pneumoniae and E7 from Human papilloma virus type 16)
atgaaagggggaattgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattggccagcatgctggacatggccatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaactagtggccaccatcaccatcaccattaa

SEQ ID NO: 12 - protein, 239 aa, Artificial Sequence
  Chimaeric protein (Clyta from Streptococcus pneumoniae and E7 from Human papilloma virus type 16)
MKGGIVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPELASMLDMAMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPTSGHHHHHH

SEQ ID NO: 13 - DNA, 1173 bp, Artificial Sequence
  Chimaeric protein (Clyta from Streptococcus pneumoniae and E6E7 fusion from Human papilloma virus type 16)
atgaaagggggaattgtacattcagacggctcttatccaaaagacaagtttgagaaaatcaatggcacttggtactactttgacagttcaggctatatgcttgcagaccgctggaggaagcacacagacggcaactggtactggttcgacaactcaggcgaaatggctacaggctggaagaaaatcgctgataagtggtactatttcaacgaagaaggtgccatgaagacaggctgggtcaagtacaaggacacttggtactacttagacgctaaagaaggcgccatggtatcaaatgcctttatccagtcagcggacggaacaggctggtactacctcaaaccagacggaacactggcagacaggccagaattggccagcatgctggacatggccatgtttcaggacccacaggagcgacccagaaagttaccacagttatgcacagagctgcaaacaactatacatgatataatattagaatgtgtgtactgcaagcaacagttactgcgacgtgaggtatatgactttgcttttcgggatttatgcatagtatatagagatgggaatccatatgctgtatgtgataaatgtttaaagttttattctaaaattagtgagtatagacattattgttatagtttgtatggaacaacattagaacagcaatacaacaaaccgttgtgtgatttgttaattaggtgtattaactgtcaaaagccactgtgtcctgaagaaaagcaaagacatctggacaaaaagcaaagattccataatataaggggtcggtggaccggtcgatgtatgtcttgttgcagatcatcaagaacacgtagagaaacccagctgatgcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccaactagtggccaccatcaccatcaccattaa

SEQ ID NO: 14 - protein, 390 aa, Artificial Sequence
  Chimaeric protein (Clyta from Streptococcus pneumoniae and E6E7 fusion from Human papilloma virus type 16)
MKGGIVHSDGSYPKDKFEKINGTWYYFDSSGYMLADRWRKHTDGNWYWFDNSGEMATGWKKIADKWYYFNEEGAMKTGWVKYKDTWYYLDAKEGAMVSNAFIQSADGTGWYYLKPDGTLADRPELASMLDMAMFQDPQERPRKLPQLCTELQTTIHDIILECVYCKQQLLRREVYDFAFRDLCIVYRDGNPYAVCDKCLKFYSKISEYRHYCYSLYGTTLEQQYNKPLCDLLIRCINCQKPLCPEEKQRHLDKKQRFHNIRGRWTGRCMSCCRSSRTRRETQLMHGDTPTLHEYMLDLQPETTDLYCYEQLNDSSEEEDEIDGPAGQAEPDRAHYNIVTFCCKCDSTLRLCVQSTHVDIRTLEDLLMGTLGIVCPICSQKPTSGHHHHHH

SEQ ID NO: 15 - DNA, 684 bp, Artificial Sequence
  Chimaeric protein (protein D from Haemophilius influenzae B and E7 from Human papilloma virus type 18)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgcatggacctaaggcaacattgcaagacattgtattgcatttagagccccaaaatgaaattccggttgaccttctatgtcacgagcaattaagcgactcagaggaagaaaacgatgaaatagatgaagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagactagtggccaccatcaccatcaccattaa

SEQ ID NO: 16 - protein, 227 aa, Artificial Sequence
  Chimaeric protein (protein D from Haemophilius influenzae B and E7 from Human papilloma virus type 18)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMHGPKATLQDIVLHLEPQNEIPVDLLCHEQLSDSEEENDEIDEVNHQHLPARRAEPQRHTMLCMCCKCEARIELVVESSADDLRAFQQLFLNTLSFVCPWCASQQTSGHHHHHH

SEQ ID NO: 17 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 18 - DNA, 684 bp, Artificial Sequence
  Chimaeric protein (protein D from Haemophilius influenzae B and mutated E7 from Human papilloma virus type 18)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggccatgcatggacctaaggcaacattgcaagacattgtattgcatttagagccccaaaatgaaattccggttgaccttctaggtcaccagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagactagtggccaccatcaccatcaccattaa

SEQ ID NO: 19 - protein, 227 aa, Artificial Sequence
  Chimaeric protein (protein D from Haemophilius influenzae B and mutated E7 from Human papilloma virus type 18)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMAMHGPKATLQDIVLHLEPQNEIPVDLLGHQQLSDSEEENDEIDGVNHQHLPARRAEPQRHTMLCMCCKCEARIELVVESSADDLRAFQQLFLNTLSFVCPWCASQQTSGHHHHHH

SEQ ID NO: 20 - DNA, 837 bp, Artificial Sequence
  Chimaeric protein (protein D from Haemophilius influenzae virus B and E6 from Human papilloma virus type 18)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtaactagtggccaccatcaccatcaccattaa

SEQ ID NO: 21 - protein, 278 aa, Artificial Sequence
  Chimaeric protein (protein D from Haemophilius influenzae B and E6 from Human papilloma virus type 18)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQVTSGHHHHHH

SEQ ID NO: 22 - DNA, 1152 bp, Artificial Sequence
  Chimaeric protein (protein D from Haemophilius influenzae B and E6E7 fusion from Human papilloma virus type 18)
atggatccaagcagccattcatcaaatatggcgaatacccaaatgaaatcagacaaaatcattattgctcaccgtggtgctagcggttatttaccagagcatacgttagaatctaaagcacttgcgtttgcacaacaggctgattatttagagcaagatttagcaatgactaaggatggtcgtttagtggttattcacgatcactttttagatggcttgactgatgttgcgaaaaaattcccacatcgtcatcgtaaagatggccgttactatgtcatcgactttaccttaaaagaaattcaaagtttagaaatgacagaaaactttgaaaccatggcgcgctttgaggatccaacacggcgaccctacaagctacctgatctgtgcacggaactgaacacttcactgcaagacatagaaataacctgtgtatattgcaagacagtattggaacttacagaggtatttgaatttgcatttaaagatttatttgtggtgtatagagacagtataccgcatgctgcatgccataaatgtatagatttttattctagaattagagaattaagacattattcagactctgtgtatggagacacattggaaaaactaactaacactgggttatacaatttattaataaggtgcctgcggtgccagaaaccgttgaatccagcagaaaaacttagacaccttaatgaaaaacgacgatttcacaacatagctgggcactatagaggccagtgccattcgtgctgcaaccgagcacgacaggaacgactccaacgacgcagagaaacacaagtaatgcatggacctaaggcaacattgcaagacattgtattgcatttagagccccaaaatgaaattccggttgaccttctatgtcacgagcaattaagcgactcagaggaagaaaacgatgaaatagatggagttaatcatcaacatttaccagcccgacgagccgaaccacaacgtcacacaatgttgtgtatgtgttgtaagtgtgaagccagaattgagctagtagtagaaagctcagcagacgaccttcgagcattccagcagctgtttctgaacaccctgtcctttgtgtgtccgtggtgtgcatcccagcagactagtggccaccatcaccatcaccattaa

SEQ ID NO: 23 - protein, 383 aa, Artificial Sequence
  Chimaeric protein (protein D from Haemophilius influenzae B and E6E7 fusion from Human papilloma virus type 18)
MDPSSHSSNMANTQMKSDKIIIAHRGASGYLPEHTLESKALAFAQQADYLEQDLAMTKDGRLVVIHDHFLDGLTDVAKKFPHRHRKDGRYYVIDFTLKEIQSLEMTENFETMARFEDPTRRPYKLPDLCTELNTSLQDIEITCVYCKTVLELTEVFEFAFKDLFVVYRDSIPHAACHKCIDFYSRIRELRHYSDSVYGDTLEKLTNTGLYNLLIRCLRCQKPLNPAEKLRHLNEKRRFHNIAGHYRGQCHSCCNRARQERLQRRRETQVMHGPKATLQDIVLHLEPQNEIPVDLLCHEQLSDSEEENDEIDGVNHQHLPARRAEPQRHTMLCMCCKCEARIELVVESSADDLRAFQQLFLNTLSFVCPWCASQQTSGHHHHHH

SEQ ID NO: 24 - DNA, 20 bp, Artificial Sequence
  Synthetic
tccatgacgttcctgacgtt

SEQ ID NO: 25 - DNA, 18 bp, Artificial Sequence
  Synthetic
tctcccagcgtgcgccat

SEQ ID NO: 26 - DNA, 30 bp, Artificial Sequence
  Synthetic
accgatgacgtcgccggtgacggcaccacg

SEQ ID NO: 27 - DNA, 6 bp, Artificial Sequence
  Synthetic
rrcgyy

SEQ ID NO: 28 - protein, 9 aa, Artificial Sequence
  E.coli
TSGHHHHHH